(19) United States
(12) Patent Application Publication
(10) Pub. No.: US 2017/0087225 A1
(43) Pub. Date: Mar. 30, 2017

(54) COMPOSITIONS AND METHODS FOR LATENT VIRAL TRANSCRIPTION REGULATION

(71) Applicant: Agenovir Corporation, South San Francisco, CA (US)

(72) Inventor: Stephen R. Quake, Stanford, CA (US)

(21) Appl. No.: 15/277,675

(22) Filed: Sep. 27, 2016

Related U.S. Application Data
(60) Provisional application No. 62/234,345, filed on Sep. 29, 2015.

(51) Int. Cl. A61K 38/46; A61K 47/48

(52) U.S. Cl. CPC A61K 38/465; C12Y 301/00; A61K 47/48092

(57) ABSTRACT

The invention provides compositions and methods that can be used to regulate viral transcription. Using a catalytically inactive nuclease such as deactivated Cas9, or dCas9, a guide RNA can be designed that recognizes a regulatory element within a viral nucleic acid. The dCas9 may function as an RNA-dependent DNA-binding protein that binds to a viral promoter and upregulates or down-regulates transcription. For example, the dCas9 with a viral promoter-specific gRNA may hybridize to a promoter within a viral genome within a host cell and inhibit transcription by, for example, sterically blocking recruitment of the transcription machinery.

### RELATED APPLICATION

This application claims benefit of U.S. Provisional Patent Application Ser. No. 62/234,345, filed Sep. 29, 2015, which is incorporated by reference.

### TECHNICAL FIELD

The invention relates to treating viral infections using compositions that include a non-cutting variant of a nuclease.

### BACKGROUND

Viral infections are a significant medical problem. For example, herpes is a widespread human pathogen, with more than 90% of adults having been infected. Due to latency, once infected, a host carries the herpes virus indefinitely, even when not expressing symptoms. Similarly, human papillomavirus, or HPV, is a common virus in the human population, where more than 75% of people will be infected. A particular problem is that viral infections may lead to cancer. For example, integration of HPV into host DNA is known to result in cancer, specifically cervical cancer. The Epstein-Barr virus (EBV) not only causes infectious mononucleosis (glandular fever), but is also associated with cancers such as Hodgkin's lymphoma and Burkitt's lymphoma.

Efforts are made to develop drugs that target viral proteins but those efforts have not been wholly successful. For example, when a virus is in a latent state, not actively expressing its proteins, there is nothing to target. Additionally, any effort to eradicate a viral infection is not optimal if it interferes with host cellular function. For example, an enzyme that prevents viral replication is not helpful if it interferes with genome replication in cells throughout the host.

### SUMMARY

The invention provides compositions and methods for treating viral infections. The invention provides a non-cutting endonuclease that binds to viral nucleic acid and interferes with viral regulatory functions. Preferred compositions of the invention include a non-cutting variant of a programmable nuclease such as Cas9. Cas9 is sometimes called a catalytically deactivated Cas9 or dCas9, to specifically target viral promoters or other regulatory elements involved in transcription or translation. Targeting is accomplished through the use of a targeting oligonucleotide such as a guide RNA (gRNA). The targeting oligonucleotide may be designed to recognize a regulatory element within a viral nucleic acid and guide the dCas9 to the targeted viral element.

With the gRNA, the programmable nuclease binds to the target in a sequence-specific manner and upregulates or down-regulates transcription. For example, a dCas9 with a viral promoter-specific gRNA may hybridize to a promoter within a viral genome in a host cell and inhibit transcription by, for example, sterically blocking recruitment of transcription machinery. Additionally or alternatively, dCas9 may be linked to another transcriptionally-repressive protein or domain. Within a viral genome, multiple targets may each be independently targeted for transcriptional repression.

A preferred use of compositions of the invention is to inhibit transcription, thus preventing expression of viral proteins. This can prevent the spread of the infection or slow the spread while other viral treatments work to eradicate the virus. Compositions of the invention include a programmable nuclease that is catalytically inactivated and that specifically targets a viral target. The programmable nuclease may be an RNA-guided nuclease (e.g., a CRISPR-associated nuclease, such as Cas9 or a modified Cas9 or Cpf1 or modified Cpf1, a Cas9 homolog, or hi-fi Cas9). The programmable nuclease may be a TALEN or a modified TALEN. In certain embodiments, the programmable nuclease may be a DNA-guided nuclease (e.g., a Pyrococcus furiosus Argonaute (PfAgo) or Natronobacterium gregoryi Argonaute (NgAgo).

A dCas9 may be used to repress transcription of a variety of targets independently or simultaneously through the provision of specific gRNAs. Thus a composition of the invention may include dCas9 (or nucleic acid encoding dCas9) as well as one or a plurality of gRNAs that each target a specific promoter within a viral genome.

Additionally or alternatively, compositions of the invention may be used to up-regulate transcription. For example, dCas9 may be linked to a transcriptionally activating domain (e.g., that recruits transcription factors) that helps up-regulate transcription. Up-regulating transcription may be useful where an antiviral agent is provided encoded within a plasmid because compositions of the invention may contribute to expression of the antiviral agent. If genes on the plasmid are under the control of a promoter of a virus that is being treated, then including a protein according the invention may augment a positive feedback cycle in which viral activity tends to stimulate expression of the antiviral therapeutic. By repressing transcription of viral genes or by up-regulating transcription of antiviral therapeutics, compositions of the invention may provide a valuable and effective way to treat viral infections.

In certain aspects, the invention provides compositions for treating a viral infection. Compositions include a vector comprising nucleic acid that encodes a non-cutting variant of a Cas9 enzyme (dCas9) and a targeting sequence complementary to a target in a viral genome. 2. In certain embodiments, the nucleic acid may comprise DNA. The dCas9 binds to the target in the viral genome via the targeting sequence and affects transcription of at least a portion of the viral genome. In some embodiments, the complex inhibits transcription of at least a portion of the viral genome.

A targeting sequence may be used that matches the target according to a predetermined criteria and does not match any portion of a host genome according to the predetermined criteria. The predetermined criteria may include being at least 60% complementary within a 20 nucleotide stretch and presence of a protospacer adjacent motif adjacent the 20 nucleotide stretch. In some embodiments, the host genome is a human and the targeting sequence does not match any portion of a human genome according to the predetermined criteria.

In preferred embodiments, the virus is capable of latent infection of a human host. Suitable targets include: a preC promoter in a hepatitis B virus (HBV) genome; an S1 promoter in the HBV genome; an S2 promoter in the HBV genome; and an X promoter in the HBV genome; a viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; and an HPV late promoter.

In some embodiments, the polypeptide further comprises a transcriptionally-repressive domain. The transcriptionally-repressive domain may include, for example, one or more of: Krüppel-associated box domain of Kox1; the chromo shadow domain of HP1α; and the WRPW domain of Hes1.

The composition may be provided within a carrier such that it is suitable for topical application to the human skin. In some embodiments, the nucleic acid is within a plasmid that is carried and delivered to the human skin by the carrier.

Any suitable virus can be targeted such as, for example, adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, junin virus, lassa virus, machupo virus, sabia virus, Crimean-Congo hemorrhagic fever virus, ebola virus, Marburg virus, measles virus, mumps virus, parainfluenza virus, respiratory syncytial virus, human metapnemovirus, Hendra virus, nipah virus, rabies virus, hepatitis D virus, rotavirus, orbivirus, coltivirus, or banna virus.

In some aspects, the invention provides a method for treating a viral infection. The method includes introducing into a host cell a composition comprising nucleic acid that encodes a polypeptide comprising a non-cutting variant of a Cas9 enzyme, and an RNA that includes a portion complementary to a target in a viral genome. In certain embodiments, the nucleic acid may comprise DNA. The polypeptide binds to the RNA to form a complex, and the complex hybridizes to the target in the viral genome via a targeting sequence within the RNA. The complex inhibits transcription of at least a portion of the viral genome. Preferably viral infection is a latent infection. Introducing the composition into the host cell may include delivering the composition to a local reservoir of latent infection within a human patient. The target in the viral genome may include any of a preC promoter in a hepatitis B virus (HBV) genome; an S1 promoter in the HBV genome; an S2 promoter in the HBV genome; and an X promoter in the HBV genome; the viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; or an HPV late promoter. The polypeptide may further include a transcriptionally-repressive domain such as a Krüppel-associated box domain of Kox1; the chromo shadow domain of HP1α; or a WRPW domain of Hes1.

In some embodiments, the method includes using the complex to cause upregulation of transcription within the host cell. For example, the polypeptide/gRNA complex may bind copies of the nucleic acid that encodes either or both of those components and upregulate their own further expression. Thus in some embodiments, wherein the nucleic acid is part of a plasmid, wherein the polypeptide binds to the RNA to form a complex, the complex hybridizes to the plasmid causing up-regulated transcription of at least a portion of the plasmid. In certain embodiments, an initial transcription of the plasmid within the host cell results in a positive feedback cycle in which the up-regulated transcription then increases the up-regulated transcription.

In some embodiments, the host cell is in situ within a host and the host is a mammal such as a human patient with the viral infection. Preferably, the composition is introduced into the cell in situ by delivery to tissue in a host. Introducing the composition into the host cell may include delivering the composition non systemically to a local reservoir of the viral infection in the host.

Any viral genome may be targeted such as the genome of adenovirus, herpes simplex virus, varicella-zoster virus, Epstein-Barr virus, human cytomegalovirus, human herpesvirus type 8, human papillomavirus, BK virus JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west Nile virus, rubella virus, hepatitis E virus, human immunodeficiency virus, influenza virus, guanarito virus, junin virus, lassa virus, machupo virus, sabia virus, Crimean-congo hemorrhagic fever virus, ebola virus, Marburg virus, measles virus, mumps virus, parainfluenza virus, respiratory syncytial virus, human metapnemovirus, Hendra virus, nipah virus, rabies virus, hepatitis D virus, rotavirus, orbivirus, coltivirus, or banna virus.

In other aspects, the invention provides a composition for treating an infection by a virus. The composition includes nucleic acid that encodes: a polypeptide comprising a non-cutting variant of a Cas9 enzyme; and an RNA that includes a targeting sequence complementary to a portion of the nucleic acid. The nucleic acid may comprise an mRNA including a 5′ cap. In certain embodiments, the nucleic acid may comprise DNA. When the composition is introduced into a cell infected by a virus: the polypeptide and the RNA are expressed; the polypeptide binds to the RNA to form a complex; and the complex hybridizes to the target in the nucleic acid and affects transcription of the nucleic acid. The nucleic acid may be part of a plasmid, and hybridization of the complex to the plasmid causes up-regulated transcription of at least a portion of the plasmid. An initial transcription of the nucleic acid within the infected cell may result in a positive feedback cycle in which the up-regulated transcription then increases the up-regulated transcription. Preferably, the targeting sequence matches the target according to a predetermined criteria and does not match any portion of a host genome according to the predetermined criteria. The nucleic acid may further encodes a promoter from a genome of a virus. In some embodiments, the complex up-regulates transcription within a host cell infected by the virus.

In certain aspects, the invention provides compositions for treating a viral infection that include a polypeptide comprising a non-cutting variant of a Cas9 enzyme and a targeting oligonucleotide complementary to a target in a viral genome. When introduced into a cell infected by the virus, the polypeptide forms a complex with the targeting oligonucleotide, the complex hybridizes to the target in the viral genome via the targeting oligonucleotide, and affects transcription of at least a portion of the viral genome. In certain embodiments, the targeting oligonucleotide comprises RNA and is complexed with the polypeptide in a ribonucleoprotein (RNP). Preferably, the complex inhibits transcription of at least a portion of the viral genome. In some embodiments, the targeting oligonucleotide comprises an RNA with a portion that matches the target according to a predetermined criteria and does not match any portion of a host genome according to the predetermined criteria (e.g., the predetermined criteria may include being at least 60% complementary within a 20 nucleotide stretch and presence of a protospacer adjacent motif adjacent the 20 nucleotide stretch). Suitable targets in the viral genome may include one or more of: a preC promoter in a hepatitis B virus (HBV) genome; an S1 promoter in the HBV genome; an S2 promoter in the HBV genome; and an X promoter in the HBV genome; a viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; and an HPV late promoter. In certain embodiments, the polypeptide further comprises a transcriptionally-repressive domain such as, for example, the Krüppel-associated box domain of Kox1, the chromo shadow domain of HP1α, or the WRPW domain of Hes1. The composition may be provided within a carrier such that it is suitable for topical application to the human skin.

In certain aspects, the invention provides a composition for treating a viral infection. The composition includes an mRNA comprising a 5′ cap that encodes a polypeptide comprising a non-cutting variant of a Cas9 enzyme (dCas9) and an RNA that includes a targeting sequence complementary to a target in a viral genome. In certain embodiments, when the composition is introduced into a cell infected by the virus, the polypeptide is expressed; the polypeptide binds to the RNA to form a complex; and the complex hybridizes to the target in the viral genome via the targeting sequence.

The dCas9 may bind to the target in the viral genome via the targeting sequence and affects transcription of at least a portion of the viral genome. In some embodiments, the complex inhibits transcription of at least a portion of the viral genome.

A targeting sequence may be used that matches the target according to a predetermined criteria and does not match any portion of a host genome according to the predetermined criteria. The predetermined criteria may include being at least 60% complementary within a 20 nucleotide stretch and presence of a protospacer adjacent motif adjacent the 20 nucleotide stretch. In some embodiments, the host genome is a human and the targeting sequence does not match any portion of a human genome according to the predetermined criteria.

In preferred embodiments, the virus is capable of latent infection of a human host. Suitable targets include: a preC promoter in a hepatitis B virus (HBV) genome; an S1 promoter in the HBV genome; an S2 promoter in the HBV genome; and an X promoter in the HBV genome; a viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; and an HPV late promoter.

In some embodiments, the polypeptide further comprises a transcriptionally-repressive domain. The transcriptionally-repressive domain may include, for example, one or more of: Krüppel-associated box domain of Kox1; the chromo shadow domain of HP1α; and the WRPW domain of Hes1.

The composition may be provided within a carrier such that it is suitable for topical application to the human skin. In some embodiments, the nucleic acid is within a plasmid that is carried and delivered to the human skin by the carrier.

Any suitable virus can be targeted such as, for example, adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, junin virus, lassa virus, machupo virus, sabia virus, Crimean-Congo hemorrhagic fever virus, ebola virus, Marburg virus, measles virus, mumps virus, parainfluenza virus, respiratory syncytial virus, human metapnemovirus, Hendra virus, nipah virus, rabies virus, hepatitis D virus, rotavirus, orbivirus, coltivirus, or banna virus.

### BRIEF DESCRIPTION OF THE DRAWINGS

FIG. 1 illustrates a composition for treating a viral infection.

FIG. 2 shows a composition for treating an infection by a virus.

FIG. 3 illustrates a plasmid according to certain embodiments of the invention.

FIG. 4 diagrams a method for treating a viral infection.

FIG. 5 diagrams an EBV reference genome.

FIG. 6 diagrams the HBV genome.

### DETAILED DESCRIPTION

The invention provides compositions and methods for regulating the transcription of viral genes, which systems and methods may be applicable within host cells, e.g., as a treatment for a viral infection. The invention uses a moiety that binds specifically to viral nucleic acid, regulates the transcription of the viral nucleic acid, and does not affect transcription of host nucleic acid. Embodiments of the invention use a composition that includes a catalytically inactive nuclease such as Cas9 or nucleic acid that encodes the catalytically inactive nuclease.

The Cas9 nuclease can be engineered to be catalytically inactive, e.g., by introducing point mutations at catalytic residues (D10A and H840A) of the gene encoding Cas9. Such mutations render Cas9 unable to cleave dsDNA but retains the ability to target DNA. This form of the protein may be referred to as dCas9, for deactivated Cas9. The dCas9 may be provided along with a guide RNA that is specific to a target within a viral genome. The system comprising dCas9 and viral gRNA provides for regulation of viral transcription within the host. Discussion of catalytically inactive dCas9 may be found in Gilbert et al., 2013, CRISPR-mediated modular RNA-guided regulation of transcription in eukaryotes, Cell 154(2):442-51, incorporated by reference. Systems of the invention may be used for the repression or the activation of transcription of viral genetic material.

Any suitable catalytically inactive nuclease may be used. Compositions and methods of the invention may use a catalytically inactive Cas9 homolog or another CRISPR-associated nuclease, ngAgo, Cpf1, or hi-fi Cas9 that has been catalytically inactivated. The nuclease may be for example, a catalytically inactive version of Cas9, ZFNs, TALENs, Cpf1, NgAgo, or a modified programmable nuclease having an amino acid sequence substantially similar to the unmodified version, for example, a programmable nuclease having an amino acid sequence at least 90% similar to one of Cas9, ZFNs, TALENs, Cpf1, or NgAgo, or any other programmable nuclease. Programmable nucleases include zinc-finger nucleases (ZFNs), transcription activator-like effector nucleases (TALENs) and RNA-guided nucleases such as the bacterial clustered regularly interspaced short palindromic repeat (CRISPR)-Cas (CRISPR-associated) nucleases or Cpf1. Programmable nucleases also include PfAgo and NgAgo. Programmable nuclease generally refers to an enzyme that cleaves nucleic acid that can be or has been designed or engineered by human contribution so that the enzyme targets or cleaves the nucleic acid in a sequence-specific manner.

Systems of the invention may be used to repress viral transcription by methods such as blocking transcriptional initiation or elongation. This is accomplished by designing sgRNA complementary to the promoter or exonic sequences, respectively. The level of transcriptional repression for exonic sequences is strand-specific. sgRNA complementary to the non-template strand more strongly represses transcription compared to sgRNA complementary to the template strand. One hypothesis to explain this effect is from the activity of helicase, which unwinds the RNA:DNA heteroduplex ahead of RNA pol II when the sgRNA is complementary to exons of the template strand. Systems of the invention may also repress transcription via an effector domain. Fusing a repressor domain to dCas9 allows transcription to be further repressed by inducing chromatin condensation. For example, the Krüppel associated box (KRAB) domain can be fused to dCas9 to repress transcription of the target gene.

Systems of the invention may be used for activation of viral or vector transcription, e.g., by fusing a transcriptional activator to dCas9. For example, the transcriptional activator VP16 may increase gene expression significantly.

Using compositions and methods of the invention, it may be possible to silence a target gene by up to 99.99% or 100% repression. Since regulation is based on Watson-Crick base-pairing of sgRNA-DNA and an NGG PAM motif, selection of targetable sites within the genome is straightforward and flexible. Carefully defined protocols for the guide RNA are presented herein. Multiple guide RNAs can not only be used to control multiple different genes simultaneously (multiplexing gene targeting), but also to enhance the efficiency of regulating the same gene target. As an exogenous system, CRISPRi does not compete with endogenous machinery such as microRNA expression or function. Furthermore, because CRISPRi acts at the DNA level, one can target transcripts such as noncoding RNAs, microRNAs, antisense transcripts, nuclear-localized RNAs, and polymerase III transcripts. Finally, CRISPRi possesses a much larger targetable sequence space; promoters and, in theory, introns can also be targeted. For background see Larson et al., 2013, CRISPR interference (CRISPRi) for sequence-specific control of gene expression, Nature Protocols 8(11):2180-96, incorporated by reference. As used herein, guide RNA or gRNA includes the gRNA with a trans-activating RNA (tracrRNA) and the use of a single guide RNA (sgRNA). Just as the isolated gRNA for use with a tracrRNA is a species of guide RNA, so is the gRNA with the tracrRNA and so also is the sgRNA. A portion of the guide RNA that hybridizes to the target is part of the targeting sequence of the guide RNA.

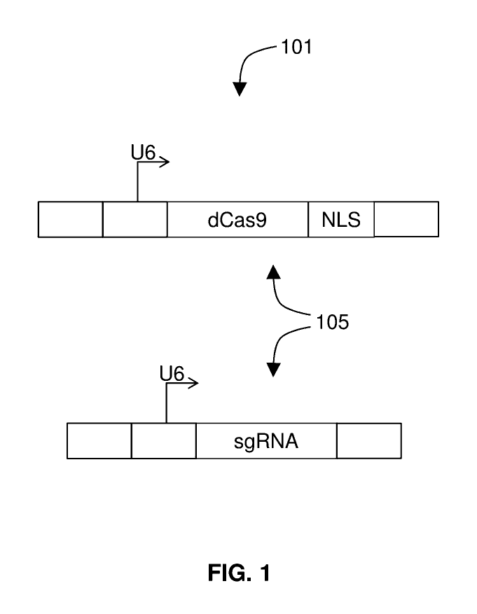

FIG. 1 illustrates a composition 101 for treating a viral infection. The composition 101 includes nucleic acid 105 that encodes a polypeptide comprising a non-cutting variant of a Cas9 enzyme and an RNA that includes a targeting sequence complementary to a target in a viral genome.

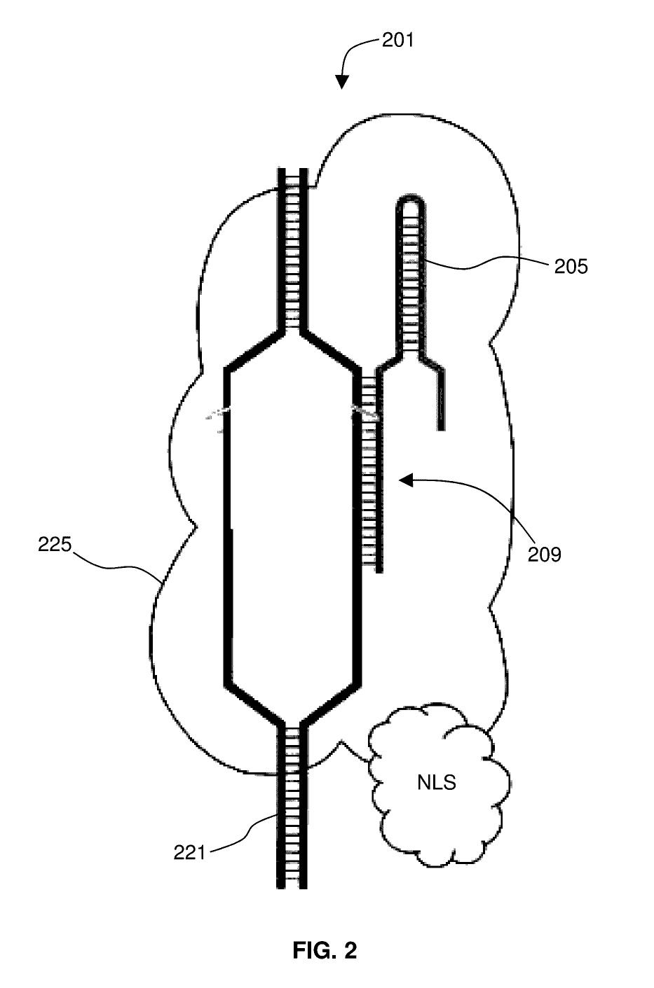

FIG. 2 shows a composition 201 for treating an infection by a virus, depicted here with the target 221. The composition 201 includes a polypeptide 225 that includes a non-cutting variant of a Cas9 enzyme and an RNA 205 that includes a targeting sequence 209 complementary to a portion of the nucleic acid 221. When the composition 201 is introduced into a cell infected by a virus, the polypeptide 225 ends up bound to the RNA 205 and hybridizes to the target in the nucleic acid and affects transcription of the nucleic acid 221.

FIG. 2 illustrates action of the composition 101 when the composition 101 is introduced into a cell infected by the virus. Within the cell, the polypeptide 225 and the RNA 205 are expressed. The polypeptide 225 binds to the RNA 205 to form a complex 201 and the complex 201 hybridizes to the target in the viral genome 221 via the targeting sequence 209. The complex 201 affects transcription of at least a portion of the viral genome 221. In some embodiments, the complex 201 inhibits transcription of at least a portion of the viral genome 221.

Using methods and compositions described herein, it may be possible to regulate the transcription of any suitable viral nucleic acid. Compositions of the invention are preferably employed to treat latent viral infections. Since latent viral infections tend not to express proteins that can be targeted by antivirals, some antiviral may not be effective. However, using compositions and methods of the invention, the target is in-fact a nucleic acid sequence and thus a latent viral infection may be targeted. For example, a gRNA may be designed that binds to a viral original of replication and may be deployed to the cell with a dCas9 (or gene for a dCas9). By means the of the gRNA, the dCas9 binds to the viral origin and inhibits any transcription or replication. Thus the latent infection never has an opportunity to reactivate. Transcription suppression with dCas9 may be very effective in combination with other antiviral treatments such as Cas9 being used to digest the viral genetic material. The dCas9 can prevent the viral from being transcribed allowing the Cas9 time and opportunity to fully digest the viral genome.

The guide RNA 205 includes a targeting sequence 209 that matches the target according to a predetermined criteria and preferably does not match any portion of a host genome according to the predetermined criteria. The targeting sequence 209 is thus, by its design, specific to a portion of the viral nucleic acid. This same sequence preferably does not appear in the host genome. Accordingly, viral nucleic acid transcription can be regulated without interfering with the host genetic material. When other systems in accordance with the invention are used, it is preferable to choose a sequence such that the system will bind to and regulate transcription of specified features or targets in the viral sequence without interfering with the host genome. Preferably, the targeting polypeptide corresponds to a nucleotide string next to a protospacer adjacent motif (PAM) (e.g., NGG, where N is any nucleotide) in the viral sequence. Preferably, the host genome lacks any region that (1) matches the nucleotide string according to a predetermined similarity criteria and (2) is also adjacent to the PAM. The predetermined similarity criteria may include, for example, a requirement of at least 12 matching nucleotides within 20 nucleotides 5′ to the PAM and may also include a requirement of at least 7 matching nucleotides within 10 nucleotides 5′ to the PAM. An annotated viral genome (e.g., from GenBank) may be used to identify features of the viral sequence and finding the nucleotide string next to a protospacer adjacent motif (PAM) in the viral sequence within a selected feature (e.g., a viral replication origin, a terminal repeat, a replication factor binding site, a promoter, a coding sequence, or a repetitive region) of the viral sequence. The viral sequence and the annotations may be obtained from a genome database.

Where multiple candidate targets are found in the viral genome, selection of the sequence to be the template for the targeting polypeptide may favor the candidate target closest to, or at the 5′ most end of, a targeted feature as the guide sequence. The selection may preferentially favor sequences with neutral (e.g., 40% to 60%) GC content. Additional background with respect to RNA-directed targeting by endonuclease is discussed in U.S. Pub. 2015/0050699; U.S. Pub. 20140356958; U.S. Pub. 2014/0349400; U.S. Pub. 2014/0342457; U.S. Pub. 2014/0295556; and U.S. Pub. 2014/0273037, the contents of each of which are incorporated by reference for all purposes. In a preferred embodiment, the predetermined similarity criteria includes being at least 60% complementary within a 20 nucleotide stretch and presence of a proto spacer adjacent motif adjacent the 20 nucleotide stretch. Also, preferably, the targeting sequence 209 does not match any portion of the human genome according to the predetermined criteria. Targets within the viral sequence 221 that may be good to target via targeting sequence 209 include, for example, the preC promoter in a hepatitis B virus (HBV) genome; the S1 promoter in the HBV genome; the S2 promoter in the HBV genome; and an X promoter in the HBV genome; the viral Cp (C promoter) in an Epstein-Barr virus genome; the minor transcript promoter region in a Kaposi's sarcoma-associated herpesvirus (KSHV) genome; the major transcript promoter in the KSHV genome; the Egr-1 promoter from a herpes-simplex virus (HSV); the ICP 4 promoter from HSV-1; the ICP 10 promoter from HSV-2; the cytomegalovirus (CMV) early enhancer element; the cytomegalovirus immediate-early promoter; the HPV early promoter; and the HPV late promoter.

Compositions and methods may be used to regulate transcription in any desired fashion. For example, in a first embodiment, dCas9 recognizes and binds to the viral nucleic acid by means of the targeting sequence 209 and down-regulates transcription by steric hindrance. That is, the dCas9 polypeptide 225 is large and bulky enough to prevent the successful assembly or operation of the transcription machinery. Further, the polypeptide 225 may include one or more additional domains or portions that contribute to transcriptional repression.

In certain embodiments, the polypeptide 225 includes or is linked to a transcriptionally-repressive domain. For example, the transcriptionally-repressive domain may include one or more of a Krüppel-associated box domain of Kox1, the chromo shadow domain of HP1α, or the WRPW domain of Hes1.

The Krüppel-associated box domain (KRAB) of Kox1 is a category of transcriptional repression domains present in approximately 400 human zinc finger protein-based transcription factors (KRAB zinc finger proteins). The KRAB domain typically consists of about 75 amino acid residues, while the minimal repression module is approximately 45 amino acid residues. See Margolin et al., 1994, Krüppel-associated boxes are potent transcriptional repression domains, PNAS 91(10):4509-13, incorporated by reference. It is predicted to function through protein-protein interactions via two amphipathic helices. The most prominent interacting protein is called TRIM28 initially visualized as SMP1, cloned as KAP1 and TIF1-beta. Over 10 independently encoded KRAB domains have been shown to be effective repressors of transcription, suggesting this activity to be a common property of the domain. The KRAB domain has initially been identified as a periodic array of leucine residues separated by six amino acids 5′ to the zinc finger region of KOX1/ZNF10 coined heptad repeat of leucines (also known as a leucine zipper). Later, this domain was named in association with the C2H2-Zinc finger proteins Krüppel associated box (KRAB). The KRAB domain is confined to genomes from tetrapod organisms. The KRAB containing C2H2-ZNF genes constitute the largest sub-family of zinc finger genes. More than half of the C2H2-ZNF genes are associated with a KRAB domain in the human genome. They are more prone to clustering and are found in large clusters on the human genome. The KRAB domain presents one of the strongest repressors in the human genome. Once the KRAB domain was fused to the tetracycline repressor (TetR), the TetR-KRAB fusion proteins were the first engineered drug-inducible repressor that worked in mammalian cells. Human genes encoding KRAB-ZFPs include KOX1/ZNF10, KOX8/ZNF708, ZNF43, ZNF184, ZNF91, HPF4, HTF10 and HTF34.

Chromo shadow domains are a protein domain that self-aggregates, causing chromatin condensation, which represses transcription. It may be particularly valuable to include a chromo shadow domain in polypeptide 225 when treating a retrovirus that is integrated into the host genome. Thus in some embodiments, compositions of the invention include a targeting sequence 209 that matches a target within a retroviral genome (such as HIV) and a polypeptide 225 that includes a sequence of dCas9 and one or more chromo shadow domains. The targeting sequence 209 and polypeptide 225 form a complex 201 within the host and bind, via the targeting sequence 209, to the integrated retroviral sequences. The chromo shadow domain(s) aggregate, condensing the chromatin, repressing transcription of the retrovirus. For this embodiment, it may be preferably that the polypeptide 225 includes a nuclear localization sequence (NLS). Thus, in some embodiments, the invention provides a vector such as a plasmid that encodes a polypeptide that includes at least one dCas9, at least one chromo shadow domain, and at least one NLS, in any suitable order. The gRNA may be encoded by that vector or another.

The WRPW domain of Hes1 refers to a Trp-Arg-Pro-Trp motif of hairy-related proteins including the Drosophila Hairy and Enhancer of Split proteins and mammalian Hes proteins. These proteins are basic helix-loop-helix (bHLH) transcriptional repressors that control cell fate decisions in both Drosophila melanogaster and mammals. Hairy-related proteins are site-specific DNA-binding proteins defined by the presence of both a repressor-specific bHLH DNA binding domain and the carboxyl-terminal WRPW (Trp-Arg-Pro-Trp) motif. These proteins act as repressors by binding to DNA sites in target gene promoters and not by interfering with activator proteins, indicating that these proteins are active repressors which should therefore have specific repression domains. See Fisher et al., 1996, Mol Cell Biol. 16:2670, incorporated by reference.

By augmenting dCas9 with a transcriptionally repressive domain, the transcriptional regulation of a composition of the invention may be strengthened.

Compositions of the method may include (e.g., be packaged within) a suitable vector including viral or non-viral vectors. In a preferred embodiment, methods and compositions of the invention provide the dCas9 and/or the gRNA encoded in a plasmid.

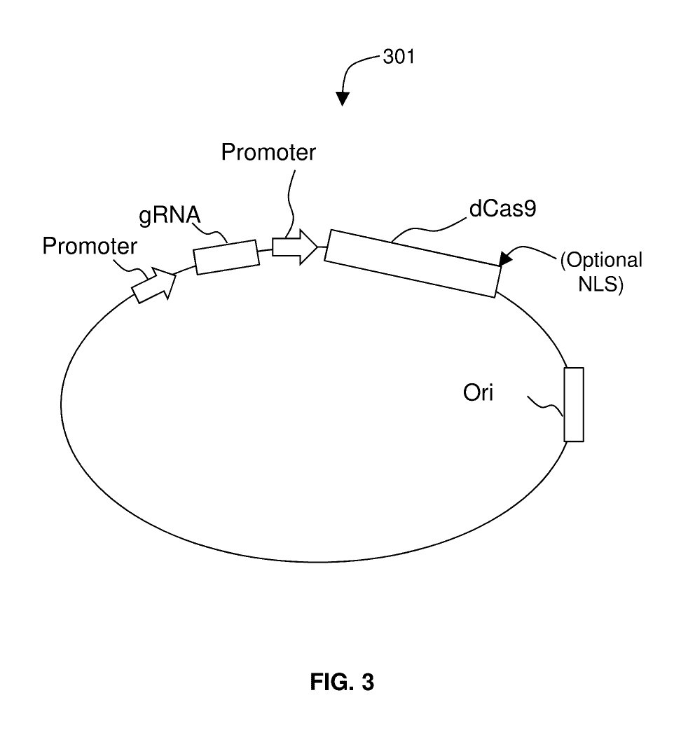

FIG. 3 illustrates a plasmid 301 that contains the nucleic acid 101. In some embodiments, the nucleic acid 101 is within the plasmid 301 and is carried and delivered to the human skin by a suitable carrier, such as any of those described or discussed above.

Additionally or alternatively, materials of the invention may be provided using a vector such as a viral vector. In some embodiment, the invention includes the use of an adeno-associated viral vector (AAV). AAVs may be used for in vivo gene delivery due to their low immunogenicity and range of serotypes allowing preferential infection of certain tissues. Where packaging the genes for dCas9 and the gRNA together (˜4.2 kb) into an AAV vector may be challenging due to the low packaging capacity of AAV (˜4.5 kb) the dCas9 and one or more gRNAs may be packaged into separate AAV vectors, increasing overall packaging capacity. The dCas9 gene may include a “shrunken” version of the original protein based on St1Cas9 from Streptococcus thermophilus and a rationally-designed truncated Cas9.

Compositions of the invention may be delivered by any suitable method include subcutaneously, transdermally, by hydrodynamic gene delivery, topically, or any other suitable method. In some embodiments, the composition 101 is provided a carrier and is suitable for topical application to the human skin. The composition may be introduced into the cell in situ by delivery to tissue in a host. Introducing the composition into the host cell may include delivering the composition non-systemically to a local reservoir of the viral infection in the host, for example, topically.

A composition of the invention may be delivered to the affected area of the skin in a acceptable topical carrier such as any acceptable formulation that can be applied to the skin surface for topical, dermal, intradermal, or transdermal delivery of a medicament. The combination of an acceptable topical carrier and the compositions described herein is termed a topical formulation of the invention. Topical formulations of the invention are prepared by mixing the composition with a topical carrier according to well-known methods in the art, for example, methods provided by standard reference texts such as, REMINGTON: THE SCIENCE AND PRACTICE OF PHARMACY 1577-1591, 1672-1673, 866-885 (Alfonso R. Gennaro ed.); Ghosh, T. K.; et al. TRANSDERMAL AND TOPICAL DRUG DELIVERY SYSTEMS (1997).

The topical carriers useful for topical delivery of the compound described herein can be any carrier known in the art for topically administering pharmaceuticals, for example, but not limited to, acceptable solvents, such as a polyalcohol or water; emulsions (either oil-in-water or water-in-oil emulsions), such as creams or lotions; micro emulsions; gels; ointments; liposomes; powders; and aqueous solutions or suspensions, such as standard ophthalmic preparations.

In certain embodiments, the topical carrier used to deliver the compositions described herein is an emulsion, gel, or ointment. Emulsions, such as creams and lotions are suitable topical formulations for use in accordance with the invention. An emulsion is a dispersed system comprising at least two immiscible phases, one phase dispersed in the other as droplets ranging in diameter from 0.1 μm to 100 μm. An emulsifying agent is typically included to improve stability.

In another embodiment, the topical carrier is a gel, for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are disclosed in REMINGTON: THE SCIENCE AND PRACTICE OF PHARMACY 1517-1518 (Alfonso R. Gennaro ed. 19th ed. 1995). Other suitable gels for use in the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002). Polymer thickeners (gelling agents) that may be used include those known to one skilled in the art, such as hydrophilic and hydro-alcoholic gelling agents frequently used in the cosmetic and pharmaceutical industries. Preferably the gelling agent comprises between about 0.2% to about 4% by weight of the composition. The agent may be a cross-linked acrylic acid polymers that are given the general adopted name carbomer. These polymers dissolve in water and form a clear or slightly hazy gel upon neutralization with a caustic material such as sodium hydroxide, potassium hydroxide, or other amine bases.

In another preferred embodiment, the topical carrier is an ointment. Ointments are oleaginous semisolids that contain little if any water. Preferably, the ointment is hydrocarbon based, such as a wax, petrolatum, or gelled mineral oil.

In another embodiment, the topical carrier used in the topical formulations of the invention is an aqueous solution or suspension, preferably, an aqueous solution. Well-known ophthalmic solutions and suspensions are suitable topical carriers for use in the invention. The pH of the aqueous topical formulations of the invention are preferably within the range of from about 6 to about 8. To stabilize the pH, preferably, an effective amount of a buffer is included. In one embodiment, the buffering agent is present in the aqueous topical formulation in an amount of from about 0.05 to about 1 weight percent of the formulation. Tonicity-adjusting agents can be included in the aqueous topical formulations of the invention. Examples of suitable tonicity-adjusting agents include, but are not limited to, sodium chloride, potassium chloride, mannitol, dextrose, glycerin, and propylene glycol. The amount of the tonicity agent can vary widely depending on the formulation's desired properties. In one embodiment, the tonicity-adjusting agent is present in the aqueous topical formulation in an amount of from about 0.5 to about 0.9 weight percent of the formulation. Preferably, the aqueous topical formulations of the invention have a viscosity in the range of from 0.015 to 0.025 Pa·s (about 15 cps to about 25 cps). The viscosity of aqueous solutions of the invention can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose.

The topical formulations of the invention can include acceptable excipients such as protectives, adsorbents, demulcents, emollients, preservatives, antioxidants, moisturizers, buffering agents, solubilizing agents, skin-penetration agents, and surfactants. Suitable protectives and adsorbents include, but are not limited to, dusting powders, zinc sterate, collodion, dimethicone, silicones, zinc carbonate, aloe vera gel and other aloe products, vitamin E oil, allatoin, glycerin, petrolatum, and zinc oxide. Suitable demulcents include, but are not limited to, benzoin, hydroxypropyl cellulose, hydroxypropyl methylcellulose, and polyvinyl alcohol. Suitable emollients include, but are not limited to, animal and vegetable fats and oils, myristyl alcohol, alum, and aluminum acetate. Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin. Chlorine dioxide (ClO2), preferably, stabilized chlorine dioxide, is a preferred preservative for use with topical formulations of the invention. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use in the invention include, but are not limited to, acetate buffers, citrate buffers, phosphate buffers, lactic acid buffers, and borate buffers. Suitable solubilizing agents include, but are not limited to, quaternary ammonium chlorides, cyclodextrins, benzyl benzoate, lecithin, and polysorbates. Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone.

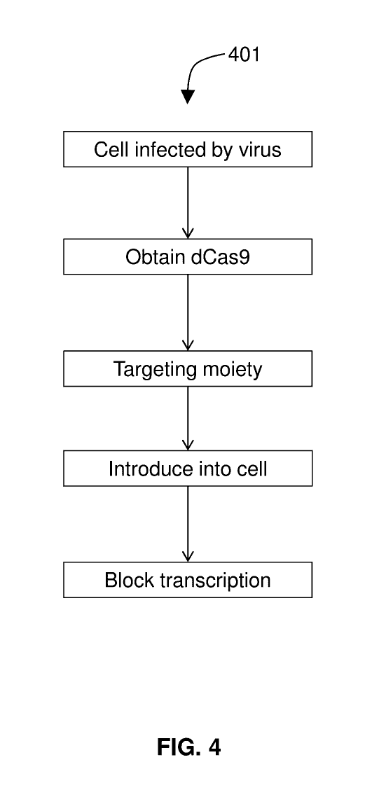

FIG. 4 diagrams a method 401 for treating a viral infection. The method 401 includes introducing into a host cell a composition 101 comprising nucleic acid 105 that encodes a polypeptide 225 that includes a non-cutting variant of a Cas9 enzyme and an RNA 205 that includes a portion 209 complementary to a target in a viral genome 221. Preferably, the polypeptide 225 binds to the RNA 205 to form a complex 201 and the complex hybridizes to the target in the viral genome 221 via a targeting sequence 209 within the RNA. In a preferred embodiment, the host cell is in situ with a host and the host is a mammal.

Preferably, a targeting sequence 209 matches the target according to a specified similarity criteria and does not match any portion of a host genome according to the similarity criteria. For example, the similarity criteria may provide that the targeting sequence and the target are at least 60% complementary within a 20 nucleotide stretch, wherein the target has a protospacer adjacent motif (PAM) adjacent the 20 nucleotide stretch. The method 401 may result in the complex 201 inhibiting transcription of at least a portion of the viral genome.

Any suitable viral genome can be targeted using the method 401. For example, the viral genome may from a virus such as adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, junin virus, lassa virus, machupo virus, sabia virus, Crimean-Congo hemorrhagic fever virus, ebola virus, Marburg virus, measles virus, mumps virus, parainfluenza virus, respiratory syncytial virus, human metapnemovirus, Hendra virus, nipah virus, rabies virus, hepatitis D virus, rotavirus, orbivirus, coltivirus, or banna virus. In a preferred embodiment, the viral genome 221 is a genome of a virus capable of latent infection of a human host. In certain embodiments, introducing the composition into the host cell includes delivering the composition to a local reservoir of latent infection within a human patient. The target in the viral genome may include such a target as a preC promoter in a hepatitis B virus (HBV) genome; an S1 promoter in the HBV genome; an S2 promoter in the HBV genome; or an X promoter in the HBV genome. In a preferred embodiment, the target in the viral genome includes the viral Cp (C promoter) in an Epstein-Barr virus (EBV) genome.

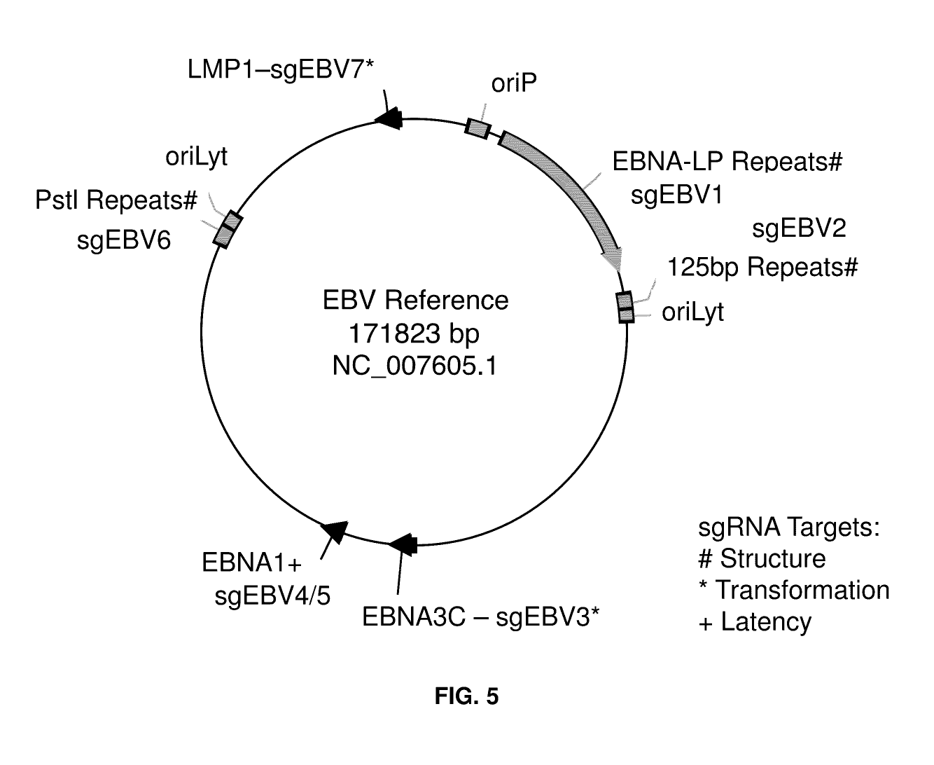

FIG. 5 diagrams an EBV reference genome. To design guide RNA targeting the EBV genome, one may refer to an EBV reference genome such as that depicted in FIG. 5. Guide RNAs may be designed that target important regions such as EBNA 1. EBNA1 is crucial for many EBV functions including gene regulation and latent genome replication. Targeting guide RNAs to either of both ends of the EBNA1 coding region may significantly interfere with transcription. As shown in FIG. 5, guide RNAs sgEBV1, 2 and 6 fall in repeat regions, increasing the probability of binding by complex 201. These “structural” targets enable systematic interference with expression of proteins important to viral function.

In certain embodiments, the target in the viral genome includes a minor transcript promoter region in a Kaposi's sarcoma-associated herpesvirus (KSHV) genome, a major transcript promoter in the KSHV genome, or both. In some embodiments, the target in the viral genome includes one or more of an Egr-1 promoter from a herpes-simplex virus (HSV); an ICP 4 promoter from HSV-1; and an ICP 10 promoter from HSV-2. In other embodiments, the target in the viral genome includes one selected from: a cytomegalovirus (CMV) early enhancer element and a cytomegalovirus immediate-early promoter. In embodiments, the target in the viral genome includes an HPV early promoter or an HPV late promoter.

In some embodiments of the invention, a composition of the invention or a complex encoded at least in part thereby is used for the up-regulation of transcription, e.g., within a cell of a host that is infected with a virus. The composition 101 includes nucleic acid 105 that encodes a polypeptide comprising a non-cutting variant of a Cas9 enzyme and an RNA that includes a targeting sequence complementary to a target in a viral genome, e.g., as shown in, for example, FIG. 1 or FIG. 3. Where the nucleic acid 105 is part of a plasmid 301, in certain embodiments, the complex 201 hybridizes to the plasmid 301 causing up-regulated transcription of at least a portion of the plasmid 301. It may be useful for an initial transcription of the nucleic acid 105 within the infected cell to contribute to a positive feedback cycle in which the up-regulated transcription then increases the up-regulated transcription. Preferably, the targeting sequence 209 matches the target according to a predetermined criteria and does not match any portion of a host genome according to the predetermined criteria. In some embodiments, the nucleic acid 105 (e.g., within plasmid 301) further encodes a promoter from a genome of a virus. By such means, the complex 225 up-regulates transcription within a host cell infected by the virus.

In some aspects and embodiments, the invention provides compositions and methods for regulating transcription using dCas9 by providing a dCas9 and a gRNA that forms a complex, wherein the complex up-regulates transcription within the host cell. For example, a plasmid 301 may be provided, wherein the polypeptide 225 binds to the RNA 205 to form a complex 201, and the complex 201 hybridizes to the plasmid 301 causing up-regulated transcription of at least a portion of the plasmid 301. Plasmid 301 may contain other elements not depicted within FIG. 3 such as regulatory sequences, genes for transcription factors or other enzymes, other genes, or combinations thereof. In some embodiments, an initial transcription of the plasmid within the host cell results in a positive feedback cycle in which the up-regulated transcription then increases the up-regulated transcription.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Targeting EBV

Burkitt's lymphoma cell lines Raji, Namalwa, and DG-75 may be obtained from ATCC and cultured in RPMI 1640 supplemented with 10% FBS and PSA, following ATCC recommendation. Human primary lung fibroblast IMR-90 may be obtained from Coriell and cultured in Advanced DMEM/F-12 supplemented with 10% FBS and PSA.

Plasmids consisting of a U6 promoter driven chimeric guide RNA (sgRNA) and a ubiquitous promoter driven dCas9 may be obtained. An EGFP marker fused after the dCas9 protein allows selection of dCas9-positive cells. A modified chimeric guide RNA design may allow for more efficient Pol-III transcription and more stable stem-loop structure (Chen B et al. (2013) Dynamic Imaging of Genomic Loci in Living Human Cells by an Optimized CRISPR/Cas System. Cell 155:1479-1491).

A modified CMV promoter with a synthetic intron (pmax) is PCR amplified from Lonza control plasmid pmax-GFP. A modified guide RNA sgRNA(F+E) is ordered from IDT. EBV replication origin oriP is PCR amplified from B95-8 transformed lymphoblastoid cell line GM12891. Standard cloning protocols may be used to clone pmax, sgRNA(F+E) and oriP to pX458, to replace the original CAG promoter, sgRNA and fl origin. EBV sgRNA may be designed based on the EBV genome shown in FIG. 5. DNA oligos are ordered from IDT. The original sgRNA place holder in pX458 serves as the negative control.

Lymphocytes are known for being resistant to lipofection, and therefore nucleofection may be used for DNA delivery into Raji cells. The Lonza pmax promoter are chosen to drive dCas9 expression as it offers strong expression within Raji cells. The Lonza Nucleofector II is used for DNA delivery. 5 million Raji or DG-75 cells are transfected with 5 ug plasmids in each 100-ul reaction. Cell line Kit V and program M-013 are used following Lonza recommendation. For IMR-90, 1 million cells are transfected with 5 ug plasmids in 100 ul Solution V, with program T-030 or X-005.

To design guide RNA targeting the EBV genome, the EBV reference genome from strain B95-8 (see FIG. 5) may be used. Six regions with seven guide RNA designs for different transcription regulation purposes may be targeted. EBNA1 is crucial for many EBV functions including gene regulation and latent genome replication. Guide RNA sgEBV4 and sgEBV5 may be targeted to both ends of the EBNA1 coding region in order to interfere with transcription of this whole region of the genome. Guide RNAs sgEBV1, 2 and 6 fall in repeat regions, so that the success rate of binding by dCas9 is increased. EBNA3C and LMP1 are essential for host cell transformation, and guide RNAs sgEBV3 and sgEBV7 are designed to target the 5′ exons of these two proteins respectively.

### Example 2

### Targeting Hepatitis B Virus (HBV)

Methods and materials of the present invention may be used to regulate transcription of specific genetic material such as a latent viral genome like the hepatitis B virus (HBV). The invention further provides for the efficient and safe delivery of nucleic acid (such as a DNA plasmid) into target cells (e.g., hepatocytes). In one embodiment, methods of the invention use hydrodynamic gene delivery to target HBV.

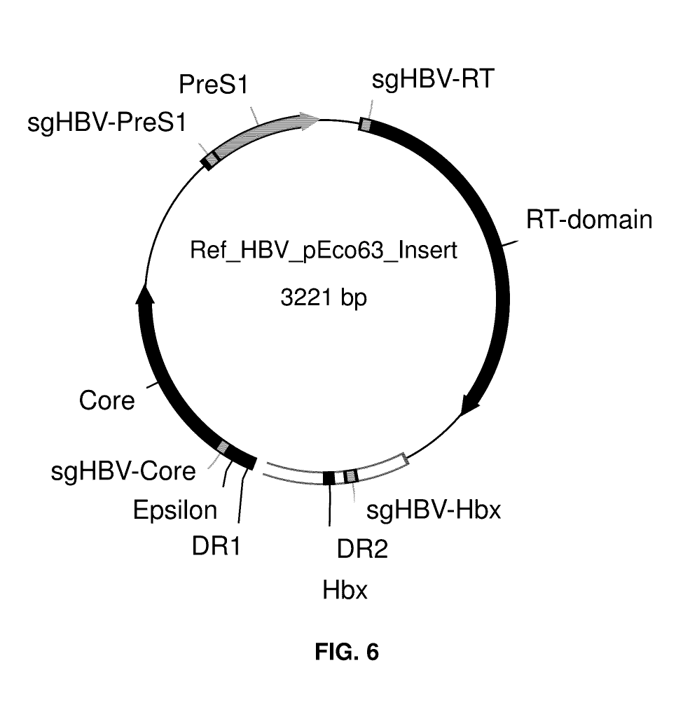

FIG. 6 diagrams the HBV genome. It may be preferable to receive annotations for the HBV genome (i.e., that identify important features of the genome) and choose a candidate for targeting by dCas9 that lies within one of those features, such as a viral replication origin, a terminal repeat, a replication factor binding site, a promoter, a coding sequence, and a repetitive region.

HBV, which is the prototype member of the family Hepadnaviridae, is a 42 nm partially double stranded DNA virus, composed of a 27 nm nucleocapsid core (HBcAg), surrounded by an outer lipoprotein coat (also called envelope) containing the surface antigen (HBsAg). The virus includes an enveloped virion containing 3 to 3.3 kb of relaxed circular, partially duplex DNA and virion-associated DNA-dependent polymerases that can repair the gap in the virion DNA template and has reverse transcriptase activities. HBV is a circular, partially double-stranded DNA virus of approximately 3200 by with four overlapping ORFs encoding the polymerase (P), core (C), surface (S) and X proteins. In infection, viral nucleocapsids enter the cell and reach the nucleus, where the viral genome is delivered. In the nucleus, second-strand DNA synthesis is completed and the gaps in both strands are repaired to yield a covalently closed circular DNA molecule that serves as a template for transcription of four viral RNAs that are 3.5, 2.4, 2.1, and 0.7 kb long. These transcripts are polyadenylated and transported to the cytoplasm, where they are translated into the viral nucleocapsid and precore antigen (C, pre-C), polymerase (P), envelope L (large), M (medium), S (small)), and transcriptional transactivating proteins (X). The envelope proteins insert themselves as integral membrane proteins into the lipid membrane of the endoplasmic reticulum (ER). The 3.5 kb species, spanning the entire genome and termed pregenomic RNA (pgRNA), is packaged together with HBV polymerase and a protein kinase into core particles where it serves as a template for reverse transcription of negative-strand DNA. The RNA to DNA conversion takes place inside the particles.

Numbering of basepairs on the HBV genome is based on the cleavage site for the restriction enzyme EcoR1 or at homologous sites, if the EcoR1 site is absent. However, other methods of numbering are also used, based on the start codon of the core protein or on the first base of the RNA pregenome. Every base pair in the HBV genome is involved in encoding at least one of the HBV protein. However, the genome also contains genetic elements that regulate levels of transcription, determine the site of polyadenylation, and even mark a specific transcript for encapsidation into the nucleocapsid. The four ORFs lead to the transcription and translation of seven different HBV proteins through use of varying in-frame start codons. For example, the small hepatitis B surface protein is generated when a ribosome begins translation at the ATG at position 155 of the adw genome. The middle hepatitis B surface protein is generated when a ribosome begins at an upstream ATG at position 3211, resulting in the addition of 55 amino acids onto the 5′ end of the protein.

ORF P occupies the majority of the genome and encodes for the hepatitis B polymerase protein. ORF S encodes the three surface proteins. ORF C encodes both the hepatitis e and core protein. ORF X encodes the hepatitis B X protein. The HBV genome contains many important promoter and signal regions necessary for viral replication to occur. The four ORFs transcription are controlled by four promoter elements (preS1, preS2, core and X), and two enhancer elements (Enh I and Enh II). All HBV transcripts share a common adenylation signal located in the region spanning 1916-1921 in the genome. Resulting transcripts range from 3.5 nucleotides to 0.9 nucleotides in length. Due to the location of the core/pregenomic promoter, the polyadenylation site is differentially utilized. The polyadenylation site is a hexanucleotide sequence (TATAAA) as opposed to the canonical eukaryotic polyadenylation signal sequence (AATAAA). The TATAAA is known to work inefficiently, suitable for differential use by HBV.

There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in-frame start (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. The function of the protein coded for by gene X is not fully understood but it is associated with the development of liver cancer. It stimulates genes that promote cell growth and inactivates growth regulating molecules.

With reference to FIG. 6, HBV starts its infection cycle by binding to the host cells with PreS1. Guide RNA against PreS1 (“sgHBV-PreS1”) locates at the 5′ end of the coding sequence. Binding by dCas9 interferes with any polymerase activity. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Transcription regulation guided by RNA against RT (“sgHBV-RT”) will interfere with RT transcription or translation. Guide RNAs sgHbx and sgCore may interfere with transcription of Hbx and HBV core protein and the whole region containing DR2-DR1-Epsilon. The four sgRNA in combination can also lead to non-transcription of HBV genome.

HBV replicates its genome by reverse transcription of an RNA intermediate. The RNA templates is first converted into single-stranded DNA species (minus-strand DNA), which is subsequently used as templates for plus-strand DNA synthesis. DNA synthesis in HBV use RNA primers for plus-strand DNA synthesis, which predominantly initiate at internal locations on the single-stranded DNA. The primer is generated via an RNase H cleavage that is a sequence independent measurement from the 5′ end of the RNA template. This 18 nucleotide RNA primer is annealed to the 3′ end of the minus-strand DNA with the 3′ end of the primer located within the 12 nucleotide direct repeat, DR1. The majority of plus-strand DNA synthesis initiates from the 12 nucleotide direct repeat, DR2, located near the other end of the minus-strand DNA as a result of primer translocation. The site of plus-strand priming has consequences. In situ priming results in a duplex linear (DL) DNA genome, whereas priming from DR2 can lead to the synthesis of a relaxed circular (RC) DNA genome following completion of a second template switch termed circularization. It remains unclear why hepadnaviruses have this added complexity for priming plus-strand DNA synthesis, but the mechanism of primer translocation is a potential therapeutic target. As viral replication is necessary for maintenance of the hepadnavirus (including the human pathogen, hepatitis B virus) chronic carrier state, understanding replication and uncovering therapeutic targets is critical for limiting disease in carriers.

In some embodiments, systems and methods of the invention target the HBV genome by finding a nucleotide string within a feature such as PreS1. Guide RNA against PreS1 locates at the 5′ end of the coding sequence. Thus it is a good candidate for targeting because it represents one of the 5′-most targets in the coding sequence and dCas9 may prevent any transcription.

HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Where dCas9 is guided by RNA against RT, RT transcription/translation may be interfered with. FIG. 6 shows key parts in the HBV genome targeted by CRISPR guide RNAs. To achieve the transcriptional regulation in cells, expression plasmids coding dCas9 and guide RNAs are delivered to cells of interest (e.g., cells carrying HBV DNA).

What is claimed is:

1. A composition for treating a viral infection, the composition comprising:
nucleic acid that encodes
a polypeptide comprising a non-cutting variant of a programmable nuclease, and
a targeting oligo that includes a targeting sequence complementary to a target in a viral genome.

2. The composition of claim 1, wherein the programmable nuclease is Cas9 and the targeting oligo is a guide RNA.

3. The composition of claim 2, wherein when the composition is introduced into a cell infected by the virus:
the polypeptide and the guide RNA are expressed;
the polypeptide binds to the guide RNA to form a complex; and
the complex hybridizes to the target in the viral genome via the targeting sequence.

4. The composition of claim 3, wherein the complex affects transcription of at least a portion of the viral genome.

5. The composition of claim 4, wherein the complex inhibits transcription of at least a portion of the viral genome.

6. The composition of claim 5, wherein the targeting sequence matches the target according to a predetermined criteria and does not match any portion of a host genome according to the predetermined criteria.

7. The composition of claim 6, wherein the host genome is the human genome and the targeting sequence does not match any portion of the human genome according to the predetermined criteria.

8. The composition of claim 7, wherein the target in the viral genome includes at least one selected from the group consisting of: a preC promoter in a hepatitis B virus (HBV) genome; an S1 promoter in the HBV genome; an S2 promoter in the HBV genome; and an X promoter in the HBV genome; a viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; and an HPV late promoter.

9. The composition of claim 5, wherein the polypeptide further comprises a transcriptionally-repressive domain.

10. The composition of claim 9, wherein the transcriptionally-repressive domain includes one selected from the group consisting of: Krüppel-associated box domain of Kox1; the chromo shadow domain of HP1α; and the WRPW domain of Hes1.

11. The composition of claim 4, wherein the complex up-regulates transcription within the host cell.

12. The composition of claim 1, wherein the viral genome is from a virus selected from the group consisting of adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, junin virus, lassa virus, machupo virus, sabia virus, Crimean-Congo hemorrhagic fever virus, ebola virus, Marburg virus, measles virus, mumps virus, parainfluenza virus, respiratory syncytial virus, human metapnemovirus, Hendra virus, nipah virus, rabies virus, hepatitis D virus, rotavirus, orbivirus, coltivirus, and banna virus.

13. A composition for treating a viral infection, the composition comprising:
a polypeptide comprising a non-cutting variant of a Cas9 enzyme, and
a targeting oligonucleotide complementary to a target in a viral genome.

14. The composition of claim 13, wherein when the composition is introduced into a cell infected by the virus:
the polypeptide forms a complex with the targeting oligonucleotide; and
the complex hybridizes to the target in the viral genome via the targeting oligonucleotide.

15. The composition of claim 13, wherein the targeting oligonucleotide comprises RNA and is complexed with the polypeptide in a ribonucleoprotein.

16. The composition of claim 14, wherein the complex affects transcription of at least a portion of the viral genome.

17. The composition of claim 16, wherein the target in the viral genome includes at least one selected from the group consisting of: a preC promoter in a hepatitis B virus (HBV) genome; an S1 promoter in the HBV genome; an S2 promoter in the HBV genome; and an X promoter in the HBV genome; a viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; and an HPV late promoter.

18. A composition for treating a viral infection, the composition comprising:
an mRNA comprising a 5′ cap that encodes a polypeptide comprising a non-cutting variant of a programmable nuclease; and
a targeting oligo that includes a targeting sequence complementary to a target in a viral genome.

19. The composition of claim 18, wherein the programmable nuclease is Cas9 and the targeting oligo is a guide RNA.

20. The composition of claim 18, wherein the programmable nuclease is selected from the group consisting of NgAgo, Cas9, argonaute, a Cas9 homolog, and Cpf1.

21. The composition of claim 18, wherein when the composition is introduced into a cell infected by the virus:
the polypeptide is expressed;
the polypeptide binds to the RNA to form a complex; and
the complex hybridizes to the target in the viral genome via the targeting sequence.

22. The composition of claim 18, wherein the complex affects transcription of at least a portion of the viral genome.

23. The composition of claim 18, wherein the target in the viral genome includes at least one selected from the group consisting of: a preC promoter in a hepatitis B virus (HBV) genome; an S1 promoter in the HBV genome; an S2 promoter in the HBV genome; and an X promoter in the HBV genome; a viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; and an HPV late promoter.
